(19) United States
(12) Patent Application Publication
(10) Pub. No.: US 2024/0125788 A1
(43) Pub. Date: Apr. 18, 2024

(54) METHOD OF IDENTIFYING THE SUBTYPE OF ACUTE LYMPHOBLASTIC LEUKEMIA WITH KMT2A GENE REARRANGEMENT

(71) Applicants: Uniwersytet Jagiellonski, Kraków (PL); Uniwersytet Medyczny w Lodzi, Lódz (PL)

(72) Inventors: Katarzyna MAJZNER, Kraków (PL); Patrycja LESZCZENKO, Michalowice (PL); Anna NOWAKOWSKA, Kraków (PL); Malgorzata BARANSKA, Trojanowice (PL); Justyna JAKUBOWSKA, Konstantynów Lódzki (PL); Marta ZABCZYNSKA, Lódz (PL); Agata PASTORCZAK, Lódz (PL); Kinga OSTROWSKA, Lódz (PL); Wojciech MLYNARSKI, Anielin Swedowski (PL)

(73) Assignees: Uniwersytet Jagiellonski, Kraków (PL); Uniwersytet Medyczny w Lodzi, Lódz (PL)

(21) Appl. No.: 18/380,230

(22) Filed: Oct. 16, 2023

(30) Foreign Application Priority Data
Oct. 15, 2022 (EU) 22461622.7

(51) Int. Cl. G01N 33/574; G01N 33/53; G01N 33/68

(52) U.S. Cl. CPC G01N 33/57496; G01N 33/5308; G01N 33/6851; G01N 2560/00

(57) ABSTRACT

A method of identifying the ALL molecular subtype with rearrangements within the KMT2A gene was disclosed, which consists in measuring the ratio of the intensity of the Raman 1040/1008 cm⁻¹ Raman bands in leukemia cells (lymphoblasts) isolated from the peripheral blood (when blastosis >90%) or bone marrow of patients during the diagnosis of acute lymphoblastic leukemia, and fixed with an aldehyde reagent.

### FIELD OF THE INVENTION

The invention relates to a new method for diagnosing the presence of KMT2A gene rearrangement in acute lymphoblastic leukemia (ALL) cells in samples collected from patients suspected to suffer from leukemia using Raman spectroscopy. Identification of cells with KMT2A gene rearrangement is based on the result of measuring the integral intensity ratio of two bands: 1040 and 1008 cm⁻¹ (the Raman shift position may vary ±3 cm⁻¹ bands due to the spectral resolution of the spectrometer used) observed on the Raman spectra of aldehyde fixed blood or bone marrow blasts.

### BACKGROUND OF THE INVENTION

Leukemias are a group of neoplastic diseases characterized by uncontrolled growth of precursor leukocytes with impaired physiological functions, inhibited in the process of differentiation. Leukemia cells primarily inhibit the myeloid niches, and in some cases also infiltrate non-myeloid organs, such as the lymph nodes, liver, or spleen. ALL, due to their high metabolic activity and intense proliferative processes, are effective in conventional chemotherapy, but the treatment process is long and burdening the patient. Leukemia relapses, which are the main cause of death in patients diagnosed with ALL, are an extremely important problem. The overall 5-year survival rate for acute lymphoblastic leukemia (ALL) in the paediatric population is almost 90%, while in adults it is only 60%. Relapses of leukemia, which are the main cause of death in patients diagnosed with ALL, are an extremely important problem [1]. According to statistics, in the United States alone, about 60,000 people suffer from leukemia annually, and about 24,000 die each year [2].

The most basic division of leukemias types includes acute and chronic, as well as lymphatic and myeloblastic. The type is distinguished by the type of cells that have undergone neoplastic transformation (lymphoid or myeloid lineage) or the course of the disease (acute and chronic leukemias). Leukemic cells (blasts) that are similar to each other in the histopathological picture may show different levels of chemosensitivity and have a different clinical picture. The source of this huge diversity lies in the extremely heterogeneous profile of disorders and abnormalities of the genomic leukemia cells that define the biological characteristics of cancer [3].

Immunophenotypic characteristics allows the distinction of ALL from B-cell progenitor acute lymphoblastic leukemia (BCP-ALL) and T-cell acute lymphoblastic leukemia (T-ALL) [4]. The diagnostic procedure for acute leukemia uses bone marrow cell aspirate, which undergoes morphological and cytochemical analysis, as well as immunophenotyping using flow cytometry and molecular and genetic tests. ALL is characterized by numerous genetic abnormalities that define its subtypes, which differ from each other in terms of cytomorphology, immunophenotype, chromosomal and gene aberrations, response to therapy and risk of recurrence.

As of now, nearly 20 molecular subtypes of BCP-ALL have been described, most of which have been defined by the presence of a particular fusion gene. Due to the fact that individual molecular subtypes of BCP-ALL may show a different response to chemotherapy, the detection of a specific genetic rearrangement in pathological lymphoblasts determines more intensive treatment, which in turn reduces the risk of leukemia recurrence [3,4].

TEL-AML1 [t(12;21) (q13;q22)], TCF3-PBX1 [t(1;19)(q23;p13)], BCR-ABL1 [Philadelphia chromosome (Ph) t(9;22)(q34;q11)-positive] fusion genes and rearrangement of the lysine methyltransferase 2a gene (KMT2A-r, formerly known as MLL), are the most recurrent disease-initiating genetic alterations in BCP-ALL. TEL-AML1 accounts for 25% of childhood cases and is related to an excellent prognosis. Similarly, TCF3-PBX1 subtype of ALL also shows a favourable outcome but increases the risk for ALL relapse in the central nervous system. In contrast, Ph-positive ALL, which occurs ten times more rarely in children as compared to adults, who represent 40-50%, is clearly associated with poor prognosis [7]. The unfavourable outcome of this subtype results from the constitutive activity of the BCR-ABL1 oncoprotein. One of the relatively common subtypes of BCP-ALL, which is also characterized by an aggressive course combined with a poor prognosis, is ALL with the KMT2A rearrangement.

Increasing the frequency of ALL with KMT2A-r is reported in the infant population, where it is associated with high leucocytosis and frequent involvement of the central nervous system (CNS). At the cytogenetic level, balanced chromosomal translocations involving 11q23 are characteristic for BCP-ALL with rearrangements within the KMT2A (KMT2A-r) gene. They lead to functional disorders resulting in an abnormal process of growth and differentiation within the haematopoietic system and the immunophenotype of pro-B lymphoid cells. Moreover, KMT2A-r lymphoblasts are characterized by an unusually high level of resistance to treatment with L-asparaginase and glucocorticosteroids and increased chemosensitivity to cytarabine [5]. A particularly extremely poor treatment outcomes and noticeably short overall survival for ALL-KMT2A-r concerns the infant population, where survival rates are at the level of 30-40%.

The current standard protocols for the diagnosis of haematopoietic malignancies, including acute leukemias, are based on cytogenetic and molecular testing. Many diagnostic tools, such as immunophenotyping (flow cytometry), molecular techniques (RNA/DNA sequencing) or histochemical tests, require highly specialized equipment and multidisciplinary and experienced laboratory personnel, which is associated with significant costs. Moreover, in non-reference medical facilities, precise diagnosis of a specific molecular subtype of leukemia is extremely difficult or even impossible.

Although flow cytometry is undoubtedly a quick and sensitive method, its principle of operation is based on the use of a wide panel of antibodies, which makes it an extremely expensive technique (costs related to the purchase of antibodies—the more detailed the analysis, the higher the costs). Moreover, the lack of standardization and subjective interpretation of the data are significant limitations felt more acutely in difficult applications such as minimal residual disease (MRD) monitoring.

Classic karyotyping and fluorescence in situ hybridization (FISH) allow the identification of some subtypes of leukemia based on the assessment of structural disorders (deletions, duplications, translocations, or inversions) of significant size (>10 Mbp) and numerical chromosome aberrations (hypodiploidy, hyperdiploidy). However, an undoubted and significant limitation resulting from the use of this methodology is the need to have active mitotic cells.

Considering the above, there is still demand for methods that would be enable fast, specific, simple, non-invasive, and low-cost diagnosis of ALL with KMT2A-r. The early diagnosis of this molecular subtype at the level of non-reference medical facilities is especially important.

These techniques are opposed by Raman spectroscopy (RS), which is a quick and non-destructive method providing biochemical characterization at the molecular level. It allows to obtain complete biochemical information without the need of use any tags and dyes. It was shown previously that primary BCP-ALL cells representing defined genetic subgroups can be discriminated from healthy cells based on the differences in the chemical composition using non-invasive and label-free Raman imaging [8]. The Raman spectroscopy-based approach of pathological cells analysis was previously reported, including a mixture with normal hematopoietic cells suspended in the solution [9,10] or B-ALL cell lines compared with normal B cells [11,12,14]. In work it was reported that Raman spectroscopy was not only able to differentiate leukemic cells from normal lymphocytes, but it also enabled accurate classification of B-cell acute leukemia into the different differentiation/maturation stages [13]. It was also shown that the spectra of B-cell leukemia cell lines are characterized by a lower ratio of the intensity of the DNA/protein bands in relation to the healthy cells [14].

In a paper Leszczenko et al., [8], we attempt to characterize BCP-ALL cells being at the same stage of maturation (pre-B), depending on the presence of the specific genetic abnormalities, including BCR-ABL1, TEL-AML1, TCF3-PBX1 gene fusions. The clinical usefulness of Raman imaging and spectroscopic markers for the characterization and identification of leukemic cells compared to normal B cells were shown. While we have achieved reproducible measurements that allowed us to distinguish between normal B cells and B-ALLs, several limitations remained. We anticipated that given the low degree of biochemical variation between studied BCP-ALL leukemia subtypes in terms of general protein and lipid composition, it would be difficult to discriminate them by means of unsupervised chemometric methods, such as e.g., PCA. Therefore, to identify reliable and robust spectral differences between ALL subtypes, a more advanced prediction model based on deep learning and a supervised approach is needed.

Unexpectedly, during further studied a method for specific identification of one of the ALL molecular subtype was obtained in this invention. Developed Raman-based method uses a simple chemometric approach of spectra analysis that allow to identify the presence of ALL blasts with KMT2A-r.

### SUMMARY OF THE INVENTION

The subject of the invention is a method for the identification of an aggressive acute lymphoblastic leukemia subtype with KMT2A gene rearrangement in blood and bone marrow mononuclear cell samples collected from patients during the standard diagnostic procedure of leukemia as a screening approach for quick identification of patients requiring intensified treatment.

The invention relates to a method for identifying an acute lymphoblastic leukemia subtype with a KMT2A gene rearrangement as defined in the appended claims.

The invention relates to a new and innovative method of identification of the KMT2A-r in aldehyde fixed ALL blasts. The method is based on the measuring of the integral intensity ratio of two bands present in the Raman spectra of blast found in blood or bone marrow samples collected from patients and fixed with an 0.5% aldehyde solution. These bands are 1040 and 1008 cm⁻¹, attributed to the presence of the phenylalanine (Phe). Raman band at ca. 1040 cm⁻¹ is additionally sensitive to the fixing factor of the tested samples—the aldehydes, which changes the Phe conformation due to cross-linking with proteins.

### DETAILED DESCRIPTION OF THE INVENTION

In an exemplary implementation, there is disclosed a method of determining the presence of the KMT2A fusion gene in blood and bone marrow samples collected from patients, comprising:
(a) collection a blood or marrow sample;
(b) isolation of leukemic cells (lymphoblasts);
(c) fixing the sample with a 0.2 μm filtered 0.5% aldehyde fixative;
(d) flushing the cells three times for 5 minutes with saline or PBS (phosphate buffer saline)
(e) collecting Raman spectra of the single cells from suspension in saline/PBS;
(f) calculating the Raman integral intensities ratio of 1040 cm⁻¹ to 1008 cm⁻¹ bands present in the Raman spectra of lymphoblasts collected from patients.
(g) it is confirmed that the value of 1040/1008 cm⁻¹ higher than 0.95 for the Raman bands is indicative of the presence of KMT2A-r in ALL gene.

In an advantageous embodiment, in stages (a) and (b) the method relates to samples of biological material that are cells isolated from the blood (when lymphoblasts are present in the peripheral blood) or the bone marrow of the patients.

In an advantageous embodiment, isolation of peripheral blood lymphoblasts can be used when the observed blastosis is higher than 90%.

In an advantageous embodiment, in stage (e) the measurement of the intensity ratio of the 1040/1008 cm⁻¹ bands is performed by analysing the Raman signal from spectra obtained using a confocal Raman imaging system, and the measurement of Raman imaging was performed using a water immersion objective. It is advantageous to use a Raman spectrometer with an imaging function as it allows the measurement of multiple spectra from a single cell, thus tracking and identifying variability within a sample by deriving a standard deviation value.

In an advantageous embodiment, in stage (e) the cell imaging measurement is conducted with preparations placed on a medium that does not exhibit a Raman signal in the spectral range of 900-1100 cm⁻¹, such as, for example, CaF₂, BaF₂ or ZnSe.

In an advantageous embodiment, in stage (e) the spectra are obtained by excitation of the samples using a laser with a wavelength of 532 nm and a laser power in the range of 20-30 mW.

Preferably, the measurement of the 1040/1008 cm⁻¹ integral intensity ratio that assessing the presence of the KMT2A fusion gene is performed in leukemic cells (blasts) obtained from the blood or bone marrow of patients during the routine diagnosis of the acute lymphoblastic leukemia subtype.

In an advantageous embodiment, in stage (c) the leukemic cell samples have been fixed for 10 minutes with 0.5% glutaraldehyde (GA) solution. It was confirmed that lower and higher GA concentrations is important for identification of BCP-ALL cells with KMT2A-r cells based on results from 1040/1008 cm⁻¹ intensities ratio. During the analysis of cells fixed with 0.1, 0.5 and 2.5% GA it was noticed that proposed method requires to work on cells fixed with 0.5% GA.

In an advantageous embodiment, the characteristic bands in the Raman spectra are located within the following ranges: from 980 to 1022 cm⁻¹ and from 1022 to 1055 cm⁻¹.

In an advantageous embodiment, in stage (f) it is accepted that the 1040/1008 cm⁻¹ ratio for the patients suffering from another BCP-ALL subtype are lower than 0.95.

In an advantageous embodiment, in stage (f) it is accepted that the 1040/1008 cm⁻¹ ratio for the healthy samples (leukocytes) are lower than 0.9.

In an advantageous embodiment, in stage (f) it is accepted that the 1040/1008 cm⁻¹ ratio for the patients suffering from BCP-ALL with KMT2A-r subtype are equal or higher than 0.95.

When the 1040/1008 cm⁻¹ band intensity ratio is greater than 0.95, the presence of the KMT2A fusion gene is detected. The above-mentioned numerical values of the position of the bands may change due to the individual variability and the spectral resolution of the spectrometer by approx. 3-5 cm⁻¹.

The advantage of the described method is that it can be used to assess the presence of rearrangements within the KMT2A gene in samples obtained from patients during diagnostics, and the analysis is based on a rapid and marker-free evaluation of the recorded signal. Another advantage of the invention is the possibility to automate the protocol and conduct diagnostics by persons trained to operate the apparatus without the need for the presence of a specialist in the field of molecular spectroscopy.

An additional advantage of the method is that blood sampling is a fast and minimally invasive process. For the test, it is necessary to take a small amount of blood to isolate leukemic cells for analysis. The invention determines which lymphoblast collection procedure to be used is suitable for the proposed method.

FIG. 1 shows a scheme of the sampling protocol for measurement and analysis; FIG. 2 illustrates a comparison of mean spectra of leukemic blasts with KMT2A and TEL-AML1 rearrangements before (live cells) and after fixation with 0.5% glutaraldehyde along with an indication of the 1040 and 1008 cm⁻¹ bands; FIG. 3 shows a comparison of mean spectra of leukemic blasts with the fusion genes: (a) KMT2A-r, (b) BCR-ABL1, (c) TCF3-PBX1, (d) TEL-AML1; FIG. 4 presents the distribution of the intensity ratio values of the integral 1040/1008 cm⁻¹ bands in the above-mentioned. Three groups of test samples subjected to a fixation process with 0.5% glutaraldehyde for 10 minutes.

### DESCRIPTION OF THE DRAWINGS

FIG. 1 Schematic representation of the workflow of preparation, measurements, analysis;

FIG. 2 Comparison of averaged Raman spectra of living (unfixed) leukemia blasts with KMT2A-r and TEL-AML1 mutation and fixed with 0.5% glutaraldehyde along with an indication of the 1040 and 1008 cm⁻¹ bands. The figure illustrates impact of 0.5% GA fixation on the Raman profile of BCP-ALL cells with KMT2A-r and TEL-AML1 mutations.

FIG. 3 illustrates a comparison of mean Raman spectra of leukemia blasts with the fusion genes: BCR-ABL1, TCF3-PBX1, TEL-AML1, and KMT2A-r. Mean Raman spectra of the whole cells fixed with 0.5% GA with the standard deviation (shadow), measured using a 532 nm laser. Spectra presented in the fingerprint range of (1800−500 cm⁻¹).

FIG. 4 illustrates the distribution of the integral intensity ratio values of the 1040/1008 cm⁻¹ bands calculated for mean spectra of each measured single cell. Three groups of test samples collected from patients suffering from other often diagnosed ALL subtypes (BCR-ABL1, TCF3-PBX1, TEL-AML1) subjected to a fixation process with 0.5% glutaraldehyde for 10 minutes. Results are presented as box plots (mean±SD, whiskers indicate minimum/maximum).

### DETAILED DESCRIPTION OF THE EMBODIMENTS

Example 1. The invention is illustrated in the following embodiment, which does not limit the protection of the invention as sought.

Collection of Samples and Isolation of Leukemic Blasts

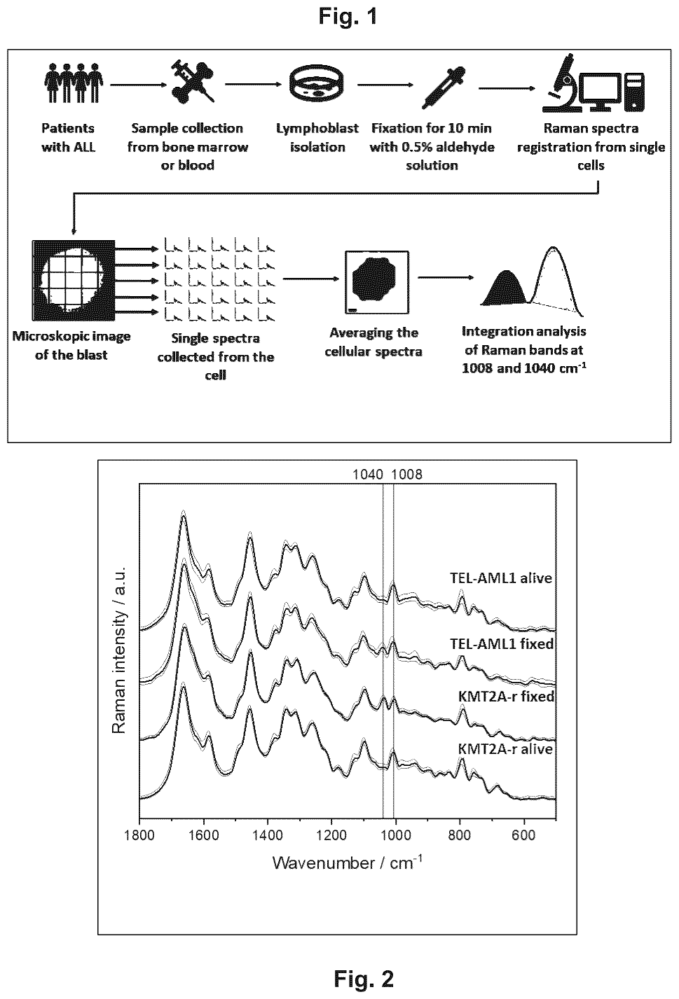

The general procedure is shown in FIG. 1. Lymphoblasts of patients with suspected B-cell acute lymphoblastic leukemia (BCP-ALL) isolated from bone marrow or peripheral blood on the day of BCP-ALL diagnosis were stored at −156° C. in liquid nitrogen gas phase. For the study samples with four typical BCP-ALL gene rearrangements were selected, i.e., BCR-ABL1, TEL-AML1, TCF3-PBX1 and KMT2A-r. Prior to Raman imaging measurements, BCP-ALL samples were removed from the gaseous liquid nitrogen phase and thawed rapidly in a water bath (37° C.). Following a flushing step with PBS (3×) at room temperature (300×g, 5 minutes), the viability of the leukemic samples was assessed using the trypan blue exclusion method.

Fixation of the Samples

Lymphoblasts were then fixed with 0.5% glutaraldehyde (GA; Sigma-Aldrich) for 10 minutes at room temperature (FIG. 1) and then washed in suspension three times with physiological saline buffer or PBS to remove any excess of the fixer. In the last step, cells after centrifugation were resuspended in physiological saline buffer (about 500 μl) and stored in Eppendorf tubes (Eppendorf Conical Tubes) at 4° C. until Raman measurements Immediately before measurements, cells were centrifuged (300×g, 5 minutes) to obtain a pellet of cells, from which approximately 200 μL was aspirated and placed directly on a calcium fluoride (CaF₂) slide.

Raman Measurements

Raman imaging measurements were performed using a WITec Alpha 300 confocal Raman microscope (Ulm, Germany) equipped with a CCD detector (Andor Technology Ltd) and 600 grooves/mm grating. For single cell measurements, a 63× water immersion objective (Zeiss W Plan-Apochromat 63×, NA=1) was used, which was then immersed in a cell suspension placed on a CaF₂ medium (25×2 mm, Crystran Ltd). In case of observing too high concentration of cells under the microscope, the suspension was diluted by adding PBS in the volume of approx. 200-300 μL. Cell morphology was assessed based on the microscopic image for each sample to measure only round cells with no visible signs of abnormalities. Each tested sample was measured as several independent biological replicates (samples from minimum 5 different patients (n=5)). About 50 cells per sample were measured for each set of cells fixed with 0.5% glutaraldehyde. Raman spectra were collected using an excitation laser with a wavelength of 532 nm. Raman images were collected with 1 μm step with an integration time of 0.5 s per spectrum and laser power of ˜20 mW (beam power measured before the objective).

Spectral Data Pre-Processing

The basic procedure for pre-processing of Raman data involved baseline correction and cosmic ray removal. For this purpose, functions available in the WITec Project Plus 5.1 software (Ulm, Germany) were used. In the first stage of the analysis of Raman images, the cosmic ray signals recorded by the CCD camera were removed (Cosmic Ray Removal, filter size—3, dynamic factor—8) and the baseline correction was applied using the polynomial function (degree 3). Then, using the same software for each recorded Raman image, k-means cluster analysis (KMCA) was performed using the Manhattan distance calculation and the Ward clustering algorithm to extract one average spectrum of the whole cell and separate the cellular spectra from the background signal (FIG. 1).

Spectral Data Analysis

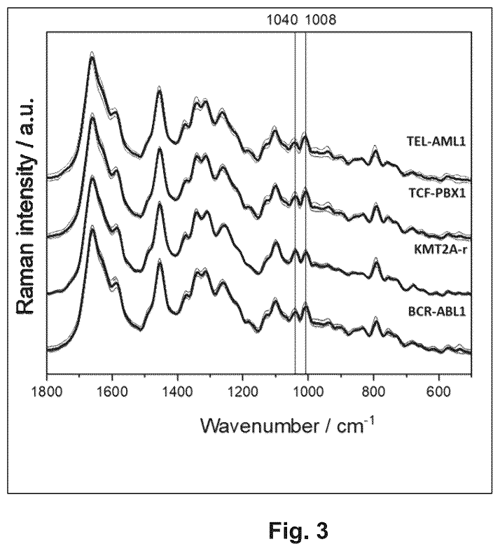

When compared Raman spectra of aldehyde fixed cells to the spectra of live cells (FIG. 2), one can observe reduced intensity of the Raman bands characteristic for hemoproteins (750, 1130, 1315, and 1585 cm⁻¹) and increasing the intensity of the band at approx. 1040 cm⁻¹. The decrease in the intensity of the bands characteristic for the cytochrome (one of the hemoproteins) is the result of its oxidation by aldehyde during the fixation process. In turn, the appearance of the 1040 cm⁻¹ band present in the spectra of fixed cells is the result of protein cross-linking as a result of a reaction with aldehyde function group. The higher intensity of the band at 1040 cm⁻¹ observed for fixed ALL cells with KMT2A-r may be related to changes in the Phe conformation in the proteins of these cells. Variations in the intensity ratios of the intensity of the bands 1040 and 1008 cm⁻¹ are also seen in the averaged spectra (FIG. 3).

Calculation of the Integral Intensity Ratio of 1040/1008 cm⁻¹

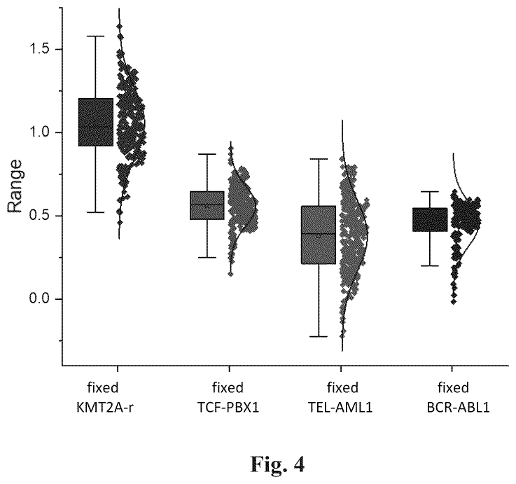

The integral intensity ratio of 1040 to 1008 cm⁻¹ Raman bands was calculated for the average spectra of each measured cell. To verify the statistical significance of the observed differences in the calculated values of the 1040/1008 cm⁻¹ ratio for all 4 ALL subtypes, analysis of variance using the ANOVA method with Tukey's test at the significance level equal to 0.05 was carried out (calculation done in the OriginPro 2020 software) (FIG. 4).

Results

As a result of the conducted experiments and data analysis, the obtained result—illustrated in FIG. 3 and FIG. 4—indicating that the value of the quantitative ratio of the 1040/1008 cm⁻¹ bands is significantly higher in the samples with the presence of KMT2A-r when compared to the spectra obtained from samples collected from patients with other molecular subtypes of BCP-ALL leukemias (FIG. 4).

In Example 1, the ratio of the average integral intensity ratio of Raman bands at 1040 to 1008 cm⁻¹ calculated from Raman spectra of samples from patients diagnosed for BCP-ALL with a rearrangement within the KMT2A gene is greater than 0.95 (FIG. 4) when cells are fixed with 0.5% GA.

Annotation

The experiments were conducted with the consent of the Bioethics Committee of the Medical University of Lodz no. RNN/270/19/KE (extension KE/30/21) of May 14, 2019. Blood from volunteers and bone marrow from patients were collected after obtaining informed consent.

### REFERENCE LITERATURE

1. Konturek, S.; Brzozowski, T. Fizjologia czlowieka. Tom 1. Fizjologia ogólna, krew i miȩśnie; Wȩgierska, D., Ed.; Wydanie V I.; Wydawnictwo Uniwersytetu Jagiellońskiego: Kraków, 2003; ISBN 83-233-1628-7.
2. https://seer.cancer.gov/statfacts/html/leuks.html Available online: https://seer.cancer.gov/statfacts/html/leuks.html.
3. Baiocchi, R. A. Classification of malignant lymphoid disorders. In Williams Hematology; McGraw-Hill Education, 2016; pp. 1493-1503 ISBN 978-0-07-183301-1.
4. Larson, R. A. Acute lymphoblastic leukemia. In Williams Hematology; McGraw-Hill Education, 2016; pp. 1505-1526 ISBN 978-0-07-183301-1.
5. Pastorczak, A.; Domka, K.; Fidyt, K.; Poprzeczko, M.; Firczuk, M. Mechanisms of immune evasion in acute lymphoblastic leukemia. Cancers (Basel). 2021, 13, 1-25, doi:10.3390/cancers13071536.
6. Winters, A. C.; Bernt, K. M. MLL-rearranged leukemias—An update on science and clinical approaches. Front. Pediatr. 2017, 5, 11-13, doi:10.3389/fped.2017.00004.
7. Roberts K. G., Mullighan C. G. The biology of B-progenitor acute lymphoblastic leukemia. Cold Spring Harb. Perspect. Med. 2020; 10:1-22, doi: 10.1101/cshperspect.a034835.
8. Leszczenko P, Borek-Dorosz A, Nowakowska A M, Adamczyk A, Kashyrskaya S, Jakubowska J, Za̧bczyńska M, Pastorczak A, Ostrowska K, Baranska M, Marzec K M, Majzner K. Towards Raman-Based Screening of Acute Lymphoblastic Leukemia-Type B (B-ALL) Subtypes. Cancers (Basel). 2021 Oct. 31; 13(21):5483. doi: 10.3390/cancers 13215483.
9. Bai Y., Yu Z., Yi S., Yan Y., Huang Z., Qiu L. Raman spectroscopy-based biomarker screening by studying the fingerprint characteristics of chronic lymphocytic leukemia and diffuse large B-cell lymphoma. J. Pharm. Biomed. Anal. 2020; 190:113514. doi: 10.1016/j.jpba.2020.113514.
10. Féré M., Gobinet C., Liu L. H., Beljebbar A., Untereiner V., Gheldof D., Chollat M., Klossa J., Chatelain B., Piot O. Implementation of a classification strategy of Raman data collected in different clinical conditions: Application to the diagnosis of chronic lymphocytic leukemia. Anal. Bioanal. Chem. 2020; 412:949-962. doi: 10.1007/s00216-019-02321-z
11. Hassoun M., Köse N., Kiselev R., Kirchberger-Tolstik T., Schie I. W., Krafft C., Popp J. Quantitation of acute monocytic leukemia cells spiked in control monocytes using surface-enhanced Raman spectroscopy. Anal. Methods. 2018; 10:2785-2791. doi: 10.1039/C8AY01046C.
12. Managò S., Valente C., Mirabelli P., De Luca A. C. Discrimination and classification of acute lymphoblastic leukemia cells by Raman spectroscopy. Opt. Sensors. 2015; 9506:95060Z. doi: 10.1117/12.2179486.
13. Managò S., Zito G., De Luca A. C. Raman microscopy based sensing of leukemia cells: A review. Opt. Laser Technol. 2018; 108:7-16. doi: 10.1016/j.optlastec.2018.06.034
14. Managò S., Valente C., Mirabelli P., Circolo D., Basile F., Corda D., De Luca A. C. A reliable Raman-spectroscopy-based approach for diagnosis, classification and follow-up of B-cell acute lymphoblastic leukemia. Sci. Rep. 2016; 6:1-13. doi: 10.1038/srep24821.

1. A method of identifying the subtype of acute lymphoblastic leukemia with KMT2A gene rearrangement, characterised in that said method comprises the following steps:
(a) providing a sample containing lymphoblasts isolated from the blood or bone marrow of the diagnosed patient, where the sample is treated with a solution of an aldehyde, preferably glutaraldehyde,
(c) recording the Raman spectra of the clinical samples from step (a);
(d) calculating the integral intensities ratio 1040/1008 cm⁻¹±3 cm⁻¹,
wherein an increase value in this ratio, compared to the value obtained for a sample from a healthy patient is indicative of the identification of an subtype of ALL with KMT2A-r.

2. The method according to claim 1, wherein in step (a) the lymphoblasts are isolated from blood or bone marrow obtained from the patient.

3. The method according to claim 1, wherein the Raman imaging of lymphoblasts is obtained from blasts isolated from peripheral blood if the number of blasts exceeds 90% of overall number of lymphocytes.

4. The method according to claim 1, wherein in step (b) the samples for measuring Raman spectra are prepared as a suspension of cells in physiological saline or PBS.

5. The method according claim 1, wherein the Raman spectra of blasts from step (b), are recorded using Raman spectrometer with excitation of a 532 nm laser equipped with a water immersive objective.

6. The method according to claim 1, wherein in step (b) single Raman spectra are recorded from at least 2 different points of at least 20 single measured cells.

7. The method according claim 1, wherein the Raman spectra of the lymphoblasts measured at stage (b) are recorded in the fingerprint range (500-1800 cm⁻¹).

8. The method according to claim 1, wherein in step (b) Phe bands are observed at 1040 and 1008 cm⁻¹±3 cm⁻¹, wherein for the presence of KMT2A gene rearrangement in cells fixed with aldehyde solution, the band intensity increases at 1040 cm⁻¹ and the band ratio of 1040/1008 cm⁻¹ increases.

9. The method according to claim 1, wherein in step (b) for cells fixed with an aldehyde solution with the presence of KMT2A-r, the calculated average value of 1040/1008 cm⁻¹ ratio±3 cm⁻¹ at stage (c) is equal or higher than 0.95.

10. The method according to claim 1, wherein at step (a) it relates to biological material, which are blood or bone marrow collected from patients at the time of ALL diagnosis.

11. The method according to claim 1, wherein at step (a) it relates to lymphoblasts isolated from blood or bone marrow.

12. The method according to claim 1, wherein it relates to identifying ALL cells with the presence of a rearrangement within the KMT2A gene.
